(19) United States
(12) Patent Application Publication
(10) Pub. No.: US 2004/0224987 A1
(43) Pub. Date: Nov. 11, 2004

(54) Crystalline form C of omeprazole sodium and the related process of its preparation, a crystalline form D of omeprazole sodium and the related process of its preparation, and a process for preparation of crystalline form a of omeprazole sodium

(75) Inventors: Manne Satyanarayana Reddy, Hyderabad (IN); Sajja Eswaraiah, Hyderabad (IN); Vetukuri Venkata Naga Kali Vara Prasada Raju, Hyderabad (IN); Rapolu Rajesh Kumar, Hyderabad (IN); Kandala Sreenadhacharyulu, Hyderabad (IN); Ganta Madhusudan Reddy, Hyderabad (IN); Boligoddu Vijayabhaskar, Hyderabad (IN); Buchi Reddy Reguri, Hyderabad (IN); Rajasekhar Kadaboina, Hyderabad (IN); Srinivas Reddy Gade, Hyderabad (IN); Babu Ireni, Hyderabad (IN)

Correspondence Address: Janet I. Cord, Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/799,433

(22) Filed: Mar. 12, 2004

(30) Foreign Application Priority Data
Mar. 13, 2003 (IN) 209/MAS/2003
Mar. 25, 2003 (IN) 254/MAS/2003
Apr. 22, 2003 (IN) 341/MAS/2003

(51) Int. Cl. A61K 31/4439; C 07D 4 3/02

(52) U.S. Cl. 514/338; 546/273.7

(57) ABSTRACT

The present invention is directed to a crystalline Form C of omeprazole sodium and the related process of its preparation, a crystalline Form D of omeprazole sodium and the related process of its preparation, and a process for preparation of crystalline Form A of omeprazole sodium.

### CROSS-REFERENCE TO RELATED APPLICATIONS

This application claims the benefit of priority to Indian Patent Application No. 209/MAS/2003 filed Mar. 13, 2003, 254/MAS/2003 filed Mar. 25, 2003 and 341/MAS/2003 filed Apr. 22, 2003, the disclosures of which are incorporated herein by reference in their entirety.

### FIELD OF INVENTION

The present invention relates to crystalline Form C of omeprazole sodium and the related process of its preparation, crystalline Form D of omeprazole sodium and the related process of its preparation, and a process for preparation of crystalline Form A of omeprazole sodium. Omeprazole sodium may be represented by the following formula I.

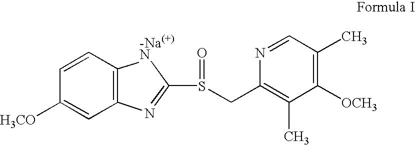

### BACKGROUND OF THE INVENTION

Polymorphic forms of the same drug are known to affect solubility, stability, flowability, fractability and compressibility of drug substances and the safety and efficacy of drug products. (see, e.g. Knapman, K. Modem Drug Discoveries, March 2000: 53). For example, many antibiotics, antibacterials, tranquilizers, etc., exhibit polymorphism and the polymorphic forms of a given drug may exhibit superior bioavailability and therefore have higher activity compared to other polymorphs. The term polymorphism includes different physical forms, crystal forms, crystalline forms and amorphous forms.

European Patent EP5129 disclosed omeprazole and its pharmaceutical acceptable salts. The specific alkaline salt of omeprazole, such as sodium salt, is disclosed in EP 124 495. The omeprazole sodium salt produced according to examples 1 and 2 of EP 124 495 is a mixture of crystal forms and amorphous material.

WO 99/00380 disclosed various forms of omeprazole sodium and those are designated as Form-A, Form-B and Form-X. These forms differ from each other in respect of physical properties, stability, spectral data and methods of preparation. Form-X of omeprazole sodium is a physically unstable and mixture of crystal forms and amorphous material. Form-B is a physically stable crystalline hydrate form.

WO 99/00380 disclosed a process for the synthesis of Form-A of omeprazole sodium, which comprises the steps of treating omeprazole with an aqueous base Na⁺B—, where in Na denotes sodium and B denotes hydroxide or alkoxide, ion exchangers, resins which releases sodium cation, at room temperature in an isopropanal solution, optionally containing water, precipitation of omeprazole Form-A at 50° C. with methanol, seeding of reaction mass with omeprazole sodium methanol wet, isolation of the product at −8° C. to −9° C. and finally drying of the product in a rotary dryer at 35 mbar with a jacket temp of 65° C., and 39-87 mbar with a jacket temperature of 50° C. for 3 days. The overall yield is 74.6%.

The resulting omeprazole sodium Form-A, which is prepared according Example: 3 in WO99/00380 is a hydrate with one to two water molecules, of which one water molecule is strongly bound in the crystal structure, while the other water molecule is easily removed by drying. The resulting dried substance containing one strongly bound water molecule is very hygroscopic and absorbs water rapidly under normal conditions.

It is also stated that omeprazole sodium Form-A produced according Example: 3 in WO 99/00380 is thermodynamically unstable, under certain strong conditions completely or partly converted into another form. Other drawbacks of this process are time consuming process, poor solubility and transmittance.

There is a constant need to prepare pharmaceutically stable crystalline forms of the active substance omeprazole sodium and an improved process for the preparation of the same, in an industrially simple and readily feasible way with high yield and proportions of residual solvents and impurities, i.e. the preparation of related substances and degradation products is low and minimal.

The claimed crystalline polymorphs of omeprazole sodium of the instant invention have been designated Form C and Form D for convenience. Form C of omeprazole sodium is relatively stable physical form and is less hygroscopic than Form A of omeprazole sodium. Form D of omeprazole sodium is thermally stable and free flowing solid. In general, the free flowing solids are recommended for pharmaceutical formulations. Hence, the present invention of the crystalline polymorph Form-D of Omeprazole sodium is well suited for pharmaceutical applications. The processes of the present invention are simple, non-hazardous and easily scalable for commercial production.

Omeprazole and its salts are useful for inhibiting gastric acid secretion as well as for providing gastrointestinal cytoprotective effects in mammals and man.

The main objective of the present invention is to provide crystalline Form C of omeprazole sodium and the related process of its preparation, crystalline Form D of omeprazole sodium and the related process of its preparation, and a process for preparation of crystalline form A of omeprazole sodium.

### SUMMARY OF THE INVENTION

The present invention is directed to a Crystalline Form C of omeprazole sodium and the related process of its preparation, a crystalline Form D of omeprazole sodium and the related process of its preparation, and a process for preparation of crystalline Form A of omeprazole sodium.

The process for the preparation of Form-A of omeprazole sodium of the present invention comprises of dissolution of omeprazole in Aqueous base, Na⁺B⁻ where in Na denotes sodium and B denotes hydroxide or alkoxide, ion exchangers, resins which releases Sodium Cation, at Room temperature in an appropiate solvent consisting of C3-C7 branched or chained hydrocarbons, C2-C7 chained or baranched ethers, cyclic ethers, lower fatty acid esters, aliphatic ketones, halogenated hydrocorbon solvents or nitrile solvents with optionally containing water, followed by neutralisation of resultant solution by an appropriate anti-solvent in which product is poorly soluble.

The present invention also relates to the process for the preparation of crystalline Form-C of omeprazole sodium which comprises dissolution of omeprazole in aqueous base, Na⁺B⁻, where in Na denotes sodium and B denotes hydroxide or alkoxide and ion exchangers, resins which releases sodium cation, at room temperature, in an appropiate solvent consisting of C3-C7 branched or chained hydrocarbons, C2-C7 chained or baranched ethers, cyclic ethers, lower fatty acid esters, aliphatic ketones, halogenated hydrocarbon solvents or acetonitrile with optionally containing water followed by neutralisation using an anti solvent from the same group in which product is poorly soluble.

The present invention also relates to the process for the preparation of crystalline Form-D of omeprazole sodium, which comprises, dissolution of omeprazole in aqueous base, Na⁺B⁻, where in Na denotes sodium and B denotes hydroxide or alkoxide and ion exchangers, resins which releases sodium cation, at room temperature, in an appropriate solvents such as acetonitrile with optionally containing water followed by neutralization using an anti solvents like halogenated hydrocarbon solvents such as Dichloro methane in which product is poorly soluble.

It is observed that Form-A, Form-C and Form-D of omeprazole respectively, obtained by above identified process are stable with relatively high yields and good purity. These processes are commercially viable and well suitable for industrial scale up.

### BRIEF DESCRIPTION OF THE ACCOMPANYING DRAWINGS

FIG. 1 is a characteristic X-ray diffraction of Form-A of omeprazole sodium.

FIG. 2 is a characteristic Infrared spectrum of Form-A of omeprazole.

FIG. 3 is a characteristic X-ray powder diffractogram of crystalline Form-C of omeprazole sodium.

FIG. 4 is a characteristic Differential Scanning Colorimetry thermogram of crystalline Form-C of omeprazole sodium.

FIG. 5 is characteristic of an Infrared spectrum of crystalline Form-C of omeprazole sodium.

FIG. 6 is characteristic of an X-ray powder diffractogram of crystalline Form-D of omeprazole sodium.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims. For purposes of the present invention, the following terms are defined below.

“Pharmaceutically acceptable” means that which is useful in preparing a pharmaceutical composition that is generally non-toxic and is not biologically undesirable and includes that which is acceptable for veterinary use and/or human pharmaceutical use.

The term “composition” includes, but is not limited to, a powder, a suspension, an emulsion and/or mixtures thereof. The term composition is intended to encompass a product containing the specified ingredients in the specified amounts, as well as any product, which results, directly or indirectly, from combination of the specified ingredients in the specified amounts. A “composition” may contain a single compound or a mixture of compounds. A “compound” is a chemical substance that includes molecules of the same chemical structure regardless of its three dimensional orientation. Thus, it may be used to indicate racemates, stereoisomers, or both.

The term “pharmaceutical composition” is intended to encompass a product including the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

The term “isolating” is used to indicate separation of the compound being isolated regardless of the purity of the isolated compound from any unwanted substance, which is present with the compound as a mixture. According to one aspect, the present invention provides a simple process for the preparation crystalline Form-A of omeprazole sodium, which can be characterized by its X-ray diffractogram, Differential Scanning Colorimetry thermogram and IR spectrum.

The X-ray diffraction pattern of Form-A of omeprazole sodium was measured on a Bruker Axe, DS Advance Powder X-ray Diffractometer with Cu K alpha-I Radiation source.

The 2-theta values and their intensity percentages of relevant peaks in X-ray powder diffraction pattern of crystalline Form-A of omeprazole sodium are shown in the Table-1.

TABLE 1

| 2-Theta | Intensity |
|---|---|
| Value (°) | I/I_{O} (%) |
| 5.64 | 100 |
| 22.53 | 17.7 |
| 12.20 | 11.1 |
| 11.17 | 10.2 |
| 16.96 | 8.1 |
| 24.57 | 5.4 |
| 25.11 | 4.7 |
| 12.86 | 4.4 |
| 28.49 | 3.3 |
| 23.15 | 3.1 |
| 13.63 | 2.8 |
| 31.50 | 2.6 |
| 30.55 | 2.3 |
| 25.96 | 2.2 |
| 16.47 | 2.1 |
| 18.84 | 2.1 |
| 21.21 | 2.1 |
| 23.68 | 2 |
| 36.99 | 1.9 |
| 20.09 | 1.7 |
| 25.40 | 1.6 |
| 34.36 | 1.4 |
| 35.33 | 1.1 |

The crystalline nature of Form-A of omeprazole sodium was characterized by its X-ray powder diffractogram substantially as depicted in FIG. (1). The moisture content of the Form-A of omeprazole sodium was characterized by its thermogravimetric analysis at a temperature range of 0-250° C. and by KF method. The thermogravimetric analysis results have shown that moisture content of the Form-A of omeprazole of present invention as 4.7%, which shows the hydrated nature of compound.

The present invention further provides the infrared data for Form-A of omeprazole sodium, which was measured by KBr-transmission method with identified significant peaks around 3439 and 2950 cm⁻¹.

The present invention provides the IR spectrum of crystalline Form-A of omeprazole sodium as depicted in FIG. (2).

Another embodiment of the present invention is to provide the process for the preparation of Form-A of omeprazole sodium, which comprises.

i) dissolution of omeprazole in an aqueous base, Na⁺B⁻ where in Na denotes sodium and B denotes hydroxide or alkoxide, Ion exchangers, resins which releases sodium cation, at room temperature, in an appropiate solvent consisting of C3-C7 branched or chained hydrocarbons, C2-C7 chained or branched ethers, cyclic ethers, lower fatty acid esters, aliphatic ketone solvents, halogenated hydrocorbon solvents or nitrile solvents with optionally containing water;

ii) neutralising the reaction mixture of step (i) using an appropriate anti solvent in which product is poorly soluble form the same group of solvents as mentioned in step (i).

iii) gently stirring the reaction mixture of step (ii) for 0-24 hrs at room temperature.

iv) cooling the reaction mixture of step (iii) till the solid mass crystallizes.

v) filtering the isolated solid of step (iv) by conventional techniques, accompanied by washing with a solvent as mentioned in step (i).

vi) drying the isolated compound of step (v) at 30-70° C. preferably at a temperature of 50-60° C. to afford Form-A of omeprazole sodium.

Thus, the present invention is directed to an improved process for the preparation of Form-A of omeprazole sodium, which is non-hydroscopic, with permissible residual solvent limits, which renders it well suited for pharmaceutical formulations. Form-A of omeprazole, obtained by the above process is thermodynamically more stable than any other prior art processes.

The crystalline Form C of omeprazole sodium of the present invention is characterized by its X-ray diffractograms, differential scanning colorimetry thermograms and IR spectra.

The X-ray diffraction pattern of Form-C of omeprazole sodium was measured on a Bruker Axe, DS Advance Powder X-ray Diffractometer with Cu K alpha-1 Radiation source. The X-ray diffractometer has shown well resolved peaks, which is a characteristic of crystalline compounds.

The 2-theta values and their intensity percentages of relevant peaks in X-ray powder diffraction pattern of crystalline Form-C of omeprazole sodium is shown in the Table-2.

TABLE 2

| 2-Theta | Intensity, |
|---|---|
| Value (°) | I/I_{O} (%) |
| 6.19 | 100.0 |
| 22.31 | 42.2 |
| 18.73 | 22.4 |
| 13.83 | 16 |
| 25.58 | 9.9 |
| 10.15 | 6.5 |
| 12.84 | 6.2 |
| 21.57 | 5.5 |
| 31.52 | 4 |
| 11.06 | 2.5 |
| 11.94 | 1.9 |
| 19.65 | 1.6 |

The present invention of crystalline Form-C of omeprazole sodium is characterized by their X-ray powder diffractogram and the relevant diffractogram is substantially as depicted in FIG. (3).

The crystalline Form-C of omeprazole sodium is also characterized by differential scanning calorimetry thermogram. The differential scanning calorimetry thermogram exhibits a significant endo peak at around 162° C. and exo peaks at around 190° C. and 208° C.

The relevant differential scanning calorimetry thermogram of crystalline Form-C of omeprazole sodium is substantially as depicted in FIG. (4).

The crystalline Form-C of omeprazole sodium of the present invention is further characterized by infrared spectrum, which is measured by KBr-transmission method. The infrared spectrum of the crystalline Form-C of omeprazole sodium is substantially as depicted in FIG. (5). The identified significant IR bands are observed around 3517, 3352 and 3162 cm⁻1. The present invention also provides a process for preparation of crystalline Form-C of omeprazole sodium.

Accordingly, the process for the preparation of crystalline Form-C of omeprazole sodium, which comprises;

i) dissolution of omeprazole in aqueous base, Na⁺B⁻, where in Na denotes sodium and B denotes hydroxide or alkoxide, ion exchangers, resins which releases sodium cation, at room temperature, in an appropriate solvent consisting of C3-C7 branched or chained hydrocarbons, C2-C7 branched or chained ethers, cyclic ethers, lower acid esters, aliphatic ketones, halogenated hydrocarbon solvents and acetonitrile with optionally containing water; neutralisation the reaction mixture of step (i) using an appropriate anti solvent in which product is poorly soluble form the same group of solvents as mentioned in step (i);

ii) optionally neutralising the reaction mixture of step (i) using an appropriate anti solvent in which product is poorly soluble from the same group of solvents as mentioned in step (i).

iii) gently stirring the reaction mixture of step (ii) for 0-24 hours preferably for 10-18 hours at 25-35° C.;

iv) optionally cooling the reaction mixture of step (iii) till the solid mass crystallizes;

v) filtering the isolated solid of step (iv) by conventional techniques, accompanied by washing with a solvent as mentioned in step (i).

vi) drying the isolated compound of step (v) at 30-70° C., preferably at a temperature of 50-60° C., to afford crystalline Form-C of omeprazole sodium.

The present invention also relates to a crystalline polymorph form of omeprazole sodium and a process for the preparation thereof designated as Form-D for convenience.

The crystalline form of omeprazole sodium of the present invention is characterized by its X-ray diffractogram. The X-ray diffraction pattern of Form-D of omeprazole sodium was measured on a Bruker Axe, DS Advance Powder X-ray Diffractometer with Cu K alpha-1 Radiation source.

The 2-theta values in X-ray powder diffraction pattern of crystalline Form-D of omeprazole sodium is shown in the Table-3.

TABLE 3

2-Theta
Value (°)
5.63
11.90
11.11
22.48
16.97
24.54
28.47
12.25
12.91
25.12
19.84
34.35
21.56
29.94
30.51
40.27
37.39
13.73
23.49
15.98
43.95

The present invention of crystalline Form-D of omeprazole sodium is characterized by their X-ray powder diffractograms and the relevant diffractogram is substantially as depicted in FIG. (6). Accordingly, the process for the preparation of crystalline Form-D of omeprazole sodium, which comprises;

i) dissolution of omeprazole in aqueous base, Na⁺B⁻, where in Na denotes sodium and B denotes hydroxide or alkoxide, ion exchangers, resins which releases sodium cation, at room temperature in an appropriate solvent such as acetonitrile with optionally containing water;

ii) neutralizing the reaction mixture of step (i) using an appropriate anti-solvent which consists of halogenated hydrocarbon solvents such as dichloromethane in which product is poorly soluble;

iii) gently stirring the reaction mixture of step (ii) for 0-10 hours preferably for 3-6 hours at a temperature of 25-35° C.;

iv) optionally cooling the reaction mixture of step (iii) till the solid mass crystallizes;

v) filtering the isolated solid of step (iv) by conventional techniques, accompanied by washing with a solvent as mentioned in step (i).

vi) drying the isolated compound of step (v) at a temperature of 30-70° C., preferably at a temperature of 50-60° C., to afford crystalline Form-D of omeprazole sodium.

It is also observed that omeprazole sodium Form-D obtained according to above process is non-hygroscopic, with permissible residual solvent limits, which renders it well suited for pharmaceutical formulations.

It is noteworthy to mention that the starting material of the above-mentioned processes, omeprazole can be prepared as per procedures known in prior art.

The present invention is described in detail with examples given below that are provided by the way of illustration only and therefore should not be construed to the limit scope of invention.

### EXAMPLES

Preparation of Crystalline Form-A of Omeprazole Sodium

### Example—1

100 grams of omeprazole was added to the mixture of 400 ml of Tetrahydrofuran, 11.6 gms of sodium hydroxide and 22 ml of water and stirred for one hour at room temperature. The resultant reaction mixture was filtered through hyflow bed and then washed with 25 ml of tetrahydrofuron. The clear filtrate was added to 1.0 lit of ethylacetate at room temperature and the reaction mixture temperature was increased to 30° C. accompanied by gentle stirring for 2 hours to crystallize the solid mass. The solid mass was then filtered under reduced pressure, washed with 50 ml of ethylacetate and dried at the temperature of 50-70° C. to afford the crystalline polymorph Form-A of omeprazole sodium.

(Weight: 108.8 grams, 97.3. %; M.C. by KF is 4.66%; Purity: 99.95%).

### Example—2

50 grams of omeprazole was added to the mixture of 200 ml of acetonitrile, 5.8 gms of sodium hydroxide and 11.0 ml of water and stirred for 40 minutes at a temperature of 20-25° C. 500 ml of acetonitrile was added to the reaction mixture at room temperature and agitated for 1-1.5 hours. The resultant reaction mixture was filtered under reduced pressure and washed with 100.0 ml of acetonitrile and dried at the temperature of 50-70° C. to afford the crystalline polymorph Form-A of omeprazole sodium.

(Weight: 53.90 grams, 96.35%; M.C. by KF is 6.2%; 99.88%).

### Example—3

25 grams of omeprazole was added to the mixture of 100 ml of dichloromethane, 3.2 grams of sodium hydroxide and 5.0 ml of water and stirred for 40 minutes at a 30° C. temperature. 200 ml+100 ml of methyl isobutyl ketone was added to the reaction mixture and stirred for 2.5 hours at 30° C. The resultant reaction mixture was filtered and then washed with 50 ml of methyl isobutyl ketone and dried at the temperature of 50-70° C. to afford the crystalline polymorph Form-A of omeprazole sodium.

(Weight: 26.8 grams, 95.88; M.C. by KF is 4.7%; Purity: 99.887%)

### Example—4

100 grams of omeprazole was added to the mixture of 100 ml of acetone, 12.8 grams of sodium hydroxide and 20.0 ml of water and stirred for 50 minutes at 30° C. The resultant reaction mixture was filtered through hyflow bed and then washed with 25 ml of acetone. Thus obtained clear filtrate was added to 800.0 lit tertiary butyl acetate at room temperature and the reaction mixture temperature was increased to 30° C. accompanied by gentle stirring for 2 hours to crystallize the solid mass. The solid mass was then filtered under reduced pressure and dried under reduced pressure to afford Form-A of omeprazole sodium and dried at a temperature of 50-70° C.

(Weight: 105.0 grams, 93.85%; Purity: 99.88%)

Preparation of Crystalline Form-C of Omeprazole Sodium:

### Example—5

100 grams of omeprazole was added to the mixture of 700 ml of tetrahydrofuran, 12.8 gms of sodium hydroxide and 20 ml of water and stirred for one hour at room temperature. The resultant reaction mixture was filtered through hyflow bed and then washed with 100 ml of tetrahydrofuran. 200 ml of ethylacetate was added to the clear filtrate at 25-35° C. accompanied by gentle stirring for 12 hours to crystallize the solid mass. The solid mass was filtered under reduced pressure, and dried at the temperature of 50-70° C. to afford the crystalline Form-C of Omeprazole sodium.

(Weight: 90.0 grams, 80.44%; M.C. by KF is 4.76%; Purity: 99.89%)

### Example—6

100 grams of omeprazole was added to the mixture of 800 ml of acetone, 12.8 gms of sodium hydroxide and 20.0 ml of water and stirred for 30 minutes at a temperature of 25-30° C. The resultant reaction mixture was filtered through hyflow bed and then washed with 100 ml of acetone. 200 ml of ethylacetate was added to the clear filtrate at 25-35° C. accompanied by gentle stirring for 13 hours to crystallize the solid mass. The solid mass was then filtered under reduced pressure, and dried at the temperature of 50-70° C. to afford the crystalline Form-C of Omeprazole sodium.

(Weight: 92.0 grams, 82.23%; M.C. by KF is 5.09%; Purity: 99.80%)

### Example—7

100 grams of omeprazole was added to the mixture of 800 ml of acetone, 11.6 gms of sodium hydroxide and 15.0 ml of water and stirred for 10 hours at a temperature of 25-30° C. to crystallize the solid mass. The resultant solid mass was filtered under reduced pressure, and dried at the temperature of 50-70° C. to afford the crystalline Form-C of omeprazole sodium.

(Weight: 100.0 grams, 89.38%; M.C. by KF is 4.99%; Purity: 99.73%)

### Example—8

20 grams of omeprazole sodium was added to the mixture of 160 ml of tetrahydrofuran, 40.0 ml of ethyl acetate and 4.0 ml of water and stirred for 8 hours at a temperature of 25-30° C. The resultant reaction mixture was filtered, washed with 20 ml of tetrahydrofuron under reduced pressure, and dried at the temperature of 50-70° C. to afford the crystalline Form-C of omeprazole sodium.

(Weight: 18.0 grams, 90%; M.C. by KF is 4.7%; Purity: 99.88%)

Preparation of Crystalline Form-D of Omeprazole Sodium:

### Example—9

100 grams of omeprazole was added to the mixture of 400 ml of acetonitrile, 11.6 grams of sodium hydroxide and 10.0 ml of water and stirred for one hour at a temperature of 25-35° C. 1000 ml of dichloromethane was added to the above clear solution at a temperature of 25-35° C. accompanied by gentle stirring for 3 hours to crystallize the solid mass. The solid mass was filtered, washed with 200 ml of acetonitrile and dried at the temperature of 50-70° C. under reduced pressure to afford the crystalline Form-D of omeprazole sodium.

(Weight: 90.0 grams, 80.44%; M.C. by KF is 5.39%)

### DETAILED DESCRIPTION OF THE ACCOMPANYING DRAWINGS

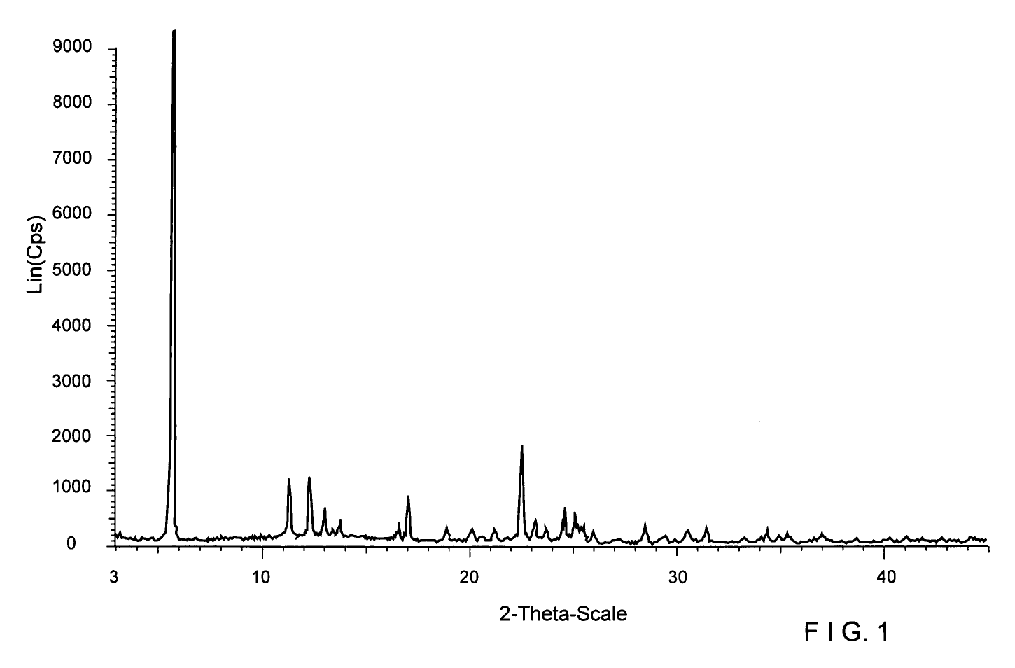

FIG. 1 is characteristic X-ray powder diffraction pattern of Form-A of omeprazole sodium. Vertical axis: Intensity (CPS); Horizontal axis: 2 Theta (degrees). The X-Ray diffraction pattern of Form-A of omeprazole sodium was measured on a Rigaku D/Max 2200 Powder Diffractometer with Cu Radiation source.

The significant 2θvalues (degrees) obtained are 5.64, 11.97, 12.19, 12.86, 13.63, 16.47, 16.95, 18.83, 20.09, 21.21, 22.52, 23.15, 23.68, 24.56, 25.10, 25.4, 25.96, 28.48, 30.55, 31.49, 34.36, 35.33 and 36.98 degrees.

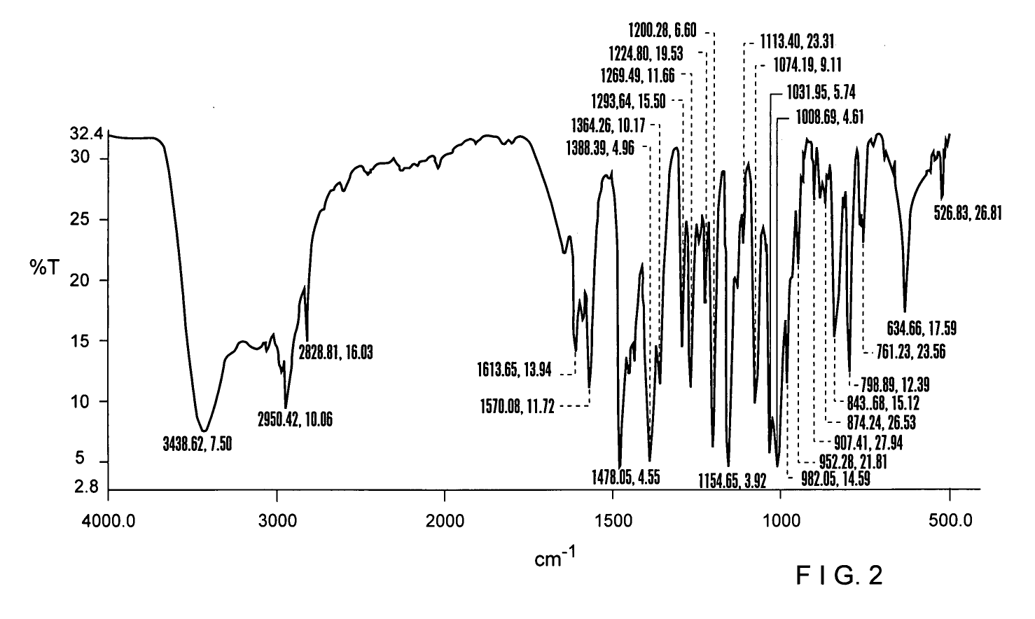

FIG. 2 is a diagram showing the results of IR spectrum of Form-A of omeprazole sodium with identified significant peaks at about 3439 and 2950 cm⁻¹.

Vertical axis: Wavelength (in Cm⁻¹); Horizontal axis: Transmission (in %).

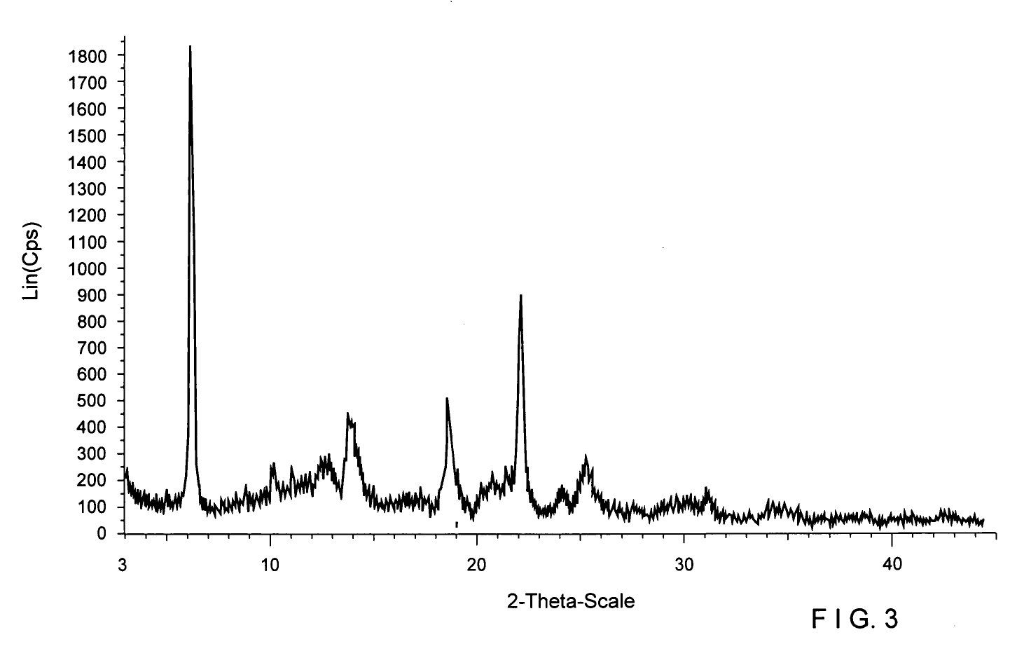

FIG. 3 is a diagram showing the results of X-ray diffraction of the novel crystalline form. Vertical axis: Intensity (CPS); Horizontal axis: 2 Theta (degrees) values.

The significant 2θ values (degrees) obtained are 6.19, 10.15, 11.06, 11.94, 12.84, 13.83, 18.73, 19.64, 21.56, 22.31, 25.58 and 31.52 degrees.

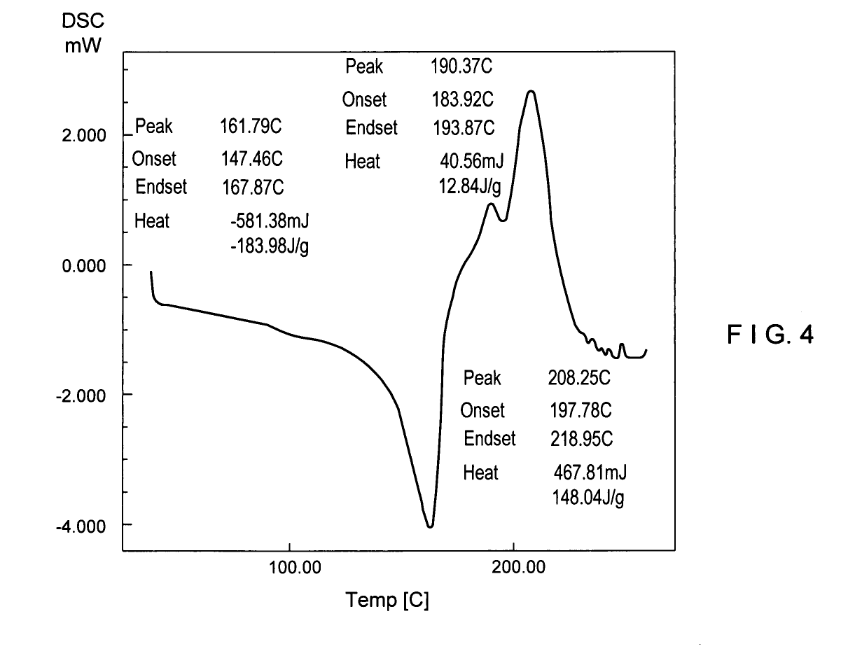

FIG. 4 is characteristic Differential Scanning Calorimetric Thermogram of novel crystalline polymorph of Form-C of Omeprazole sodium.

Vertical axis: Temperature (in ° C.); Horizontal axis: Signal (in mV).

The Differential Scanning Calorimetric Thermogram exhibits a significant endo peak at around 162° C. and exo peaks at around 190° C. and 208° C.

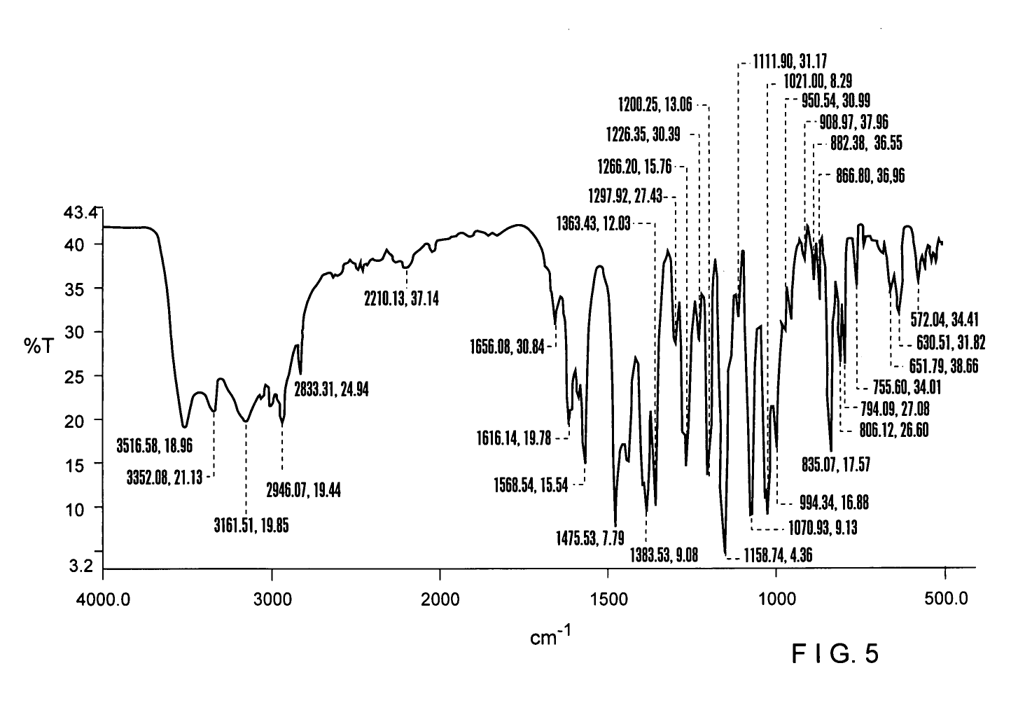

FIG. 5 is characteristic Infrared spectrum of crystalline Form-C of omeprazole sodium, with identified significant peaks at around 3517, 3352 and 3162 cm⁻1.

Vertical axis: Wavelength (in Cm⁻¹); Horizontal axis: Transmission (in %).

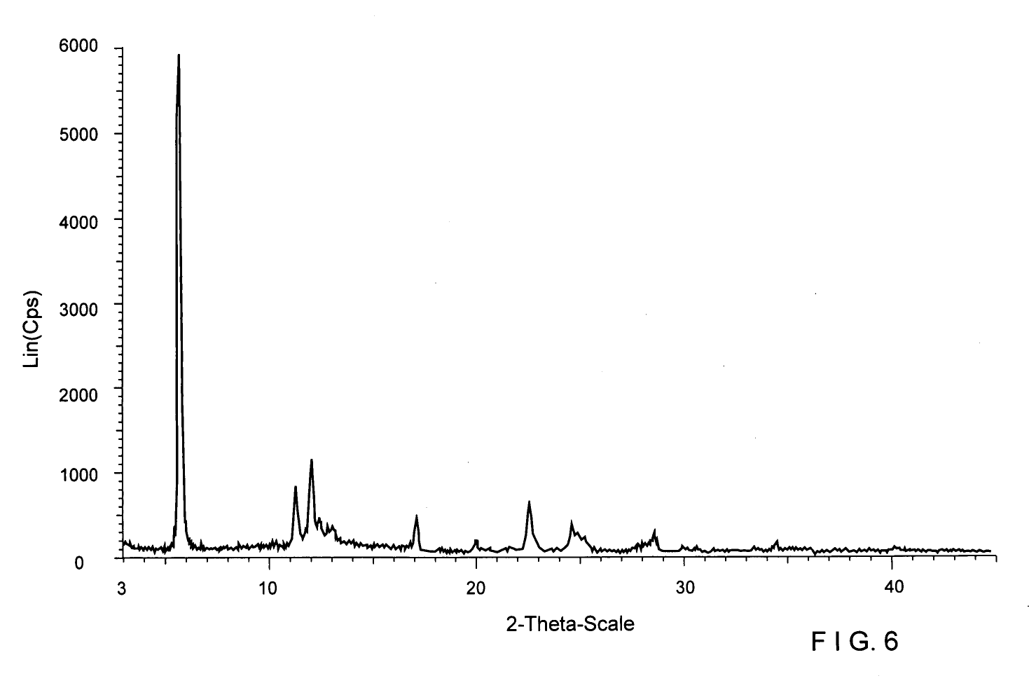

FIG. 6 is a diagram showing the results of X-ray diffraction of the crystalline Form-D of omeprazole sodium.

Vertical axis: Intensity (CPS); Horizontal axis: 2 Theta (degrees) values.

The significant 2θ value (degrees) obtained are 5.632, 11.107, 11.896, 12.249, 12.908, 13.733, 15.982, 16.974, 19.838, 21.561, 22.480, 23.487, 24.535, 25.123, 28.467, 29.937, 30.506, 34.351, 37.390, 40.269 and 43.952 degrees.

We claim:

1. An improved process for the preparation of crystalline Form-A of the sodium salt of 5-methoxy-2-[[(4-methoxy-3,5-dimethyl-2-pyridinyl)methyl]sulfinyl]-1H-benzimidazole (omeprazole sodium), which comprises;
i) dissolution of omeprazole in an aqueous base, Na⁺B⁻ where in Na denotes sodium and B denotes hydroxide or alkoxide, Ion exchangers, resins which releases sodium cation, at room temperature, in an appropiate solvent consisting of C3-C7 branched or chained hydrocarbons, C2-C7 chained or branched ethers, cyclic ethers, lower fatty acid esters, aliphatic ketone solvents, halogenated hydrocarbon solvents or nitrile solvents with optionally containing water;
ii) neutralising the reaction mixture of step (i) using an appropriate anti-solvent in which product is poorly soluble form the same group of solvents as mentioned in step (i).
iii) stirring the reaction mixture of step (ii) for 0-24 hrs at room temperature.
iv) cooling the reaction mixture of step (iii) till the solid mass crystallizes.
v) filtering the isolated solid of step (iv) by conventional techniques, accompanied by washing with a solvent as mentioned in step (i).
vi) drying the isolated compound of step (v) at 30-70° C. preferably at a temperature of 50-60° C. to afford Form-A of omeprazole sodium.

2. A process according to claim 1 of step (i) wherein preferable solvents are tetrahydrofuran, acetonitrile, ethyl acetate, acetone or dichloromethane.

3. A process according to claim 1 of step (i) where in preferable anti solvents are ethyl acetate, acetonitrile, methyl isobutyl ketone or tertiary butyl acetate.

4. The crystalline Form-C of sodium salt of 5-methoxy-2-[[(4-methoxy-3,5-dimethyl-2-pyridinyl)methyl]sulfinyl]-1H-benzimidazole (omeprazole sodium).

5. The crystalline Form-C of omeprazole sodium of claim 4 having X-ray powder diffraction pattern with peaks around 6.19, 10.15, 11.06, 11.94, 12.84, 13.83, 18.73, 19.64, 21.56, 22.31, 25.58 and 31.52 two-theta degrees.

6. The crystalline Form-C of omeprazole sodium according to claim 4, which provides X-ray diffraction pattern substantially in accordance with FIG. (1).

7. The crystalline Form-C of omeprazole sodium according to claim 4 having a differential scanning colorimetry thermogram, which exhibits a significant endo peak at around 162° C. and exo peaks at around 190° C. and 208° C.

8. The crystalline Form-C of omeprazole sodium according to claim 7 having a Differential Scanning Colorimetry thermogram substantially in accordance with FIG. (4).

9. The crystalline Form-C of omeprazole sodium according to claim 4 having an identified characteristic bands at having 3517, 3352 and 3162 cm⁻1 in infrared spectrum.

10. The crystalline Form-C of omeprazole sodium according to claim 4 having an Infrared spectrum substantially in accordance with FIG. (5).

11. A process for preparing crystalline Form-C of omeprazole sodium, which comprises;
i) dissolution of omeprazole in aqueous base, Na⁺B⁻ where in Na denotes sodium and B denotes hydroxide or alkoxide, ion exchangers, resins which releases sodium cation, at room temperature, in an appropriate solvent consisting of C3-C7 branched or chained hydrocarbons, C2-C7 branched or chained ethers, cyclic ethers, lower acid esters, aliphatic ketones, halogenated hydrocarbon solvents and acetonitrile with optionally containing water; neutralisation the reaction mixture of step (i) using an appropriate anti solvent in which Product is poorly soluble Form the same group of solvents as mentioned in step (i);
ii) optionally neutralising the reaction mixture of step (i) using an appropriate anti solvent in which product is poorly soluble from the same group of solvents as mentioned in step (i).
iii) gently stirring the reaction mixture of step (ii) for 0-24 hours preferably for 10-18 hours at 25-35° C.;
iv) optionally cooling the reaction mixture of step (iii) till the solid mass crystallizes;
v) filtering the isolated solid of step (iv) by conventional techniques, accompanied by washing with a solvent as mentioned in step (i).
vi) drying the isolated compound of step (v) at 30-70° C. preferably at a temperature of 50-60° C. to afford novel crystalline Form-C of omeprazole sodium.

12. The process according to step (i) of claim 11, wherein the preferable solvents are tetrahydrofuran and acetone.

13. A process according to claim 11 of step (i) where in preferable anti solvent is ethyl acetate.

14. The crystalline Form-D of sodium salt of 5-methoxy-2-[[(4-methoxy-3,5-dimethyl-2-pyridinyl)methyl]sulfinyl]-1H-benzimidazole (omeprazole sodium).

15. The crystalline Form-D of omeprazole sodium of claim 14 having characteristic peak at 11.896 two-theta degrees.

16. The crystalline Form-D of omeprazole sodium according to claim 14, which provides X-ray diffraction pattern substantially in accordance with FIG. (6).

17. A process for preparing crystalline Form-D of omeprazole sodium, which comprises;
vii) dissolution of omeprazole in aqueous base, Na⁺B⁻, where in Na denotes sodium and B denotes hydroxide or alkoxide, ion exchangers, resins which releases sodium cation, at room temperature in an appropriate solvent such as acetonitrile with optionally containing water;
viii) neutralising the reaction mixture of step (i) using an appropriate anti solvent which consists of halogenated hydrocarbon solvents such as dichloromethane in which product is poorly soluble;
ix) gently stirring the reaction mixture of step (ii) for 0-10 hours preferably for 3-6 hours at a temperature of 25-35° C.;
x) optionally cooling the reaction mixture of step (iii) till the solid mass crystallizes;
xi) filtering the isolated solid of step (iv) by conventional techniques, accompanied by washing with a solvent as mentioned in step (i).
xii) drying the isolated compound of step (v) at a temperature of 30-70° C. preferably at a temperature of 50-60° C. to afford novel crystalline Form-D of omeprazole sodium.

18. The process according to step (i) of claim 17, wherein the preferable solvent is acetonitrile.

19. The process according to step (i) of claim 17, wherein the preferable anti solvent is dichloromethane.
